(19) United States
(12) Patent Application Publication
(10) Pub. No.: US 2026/0144435 A1
(43) Pub. Date: May 28, 2026

(54) Medical imaging device and method for medical imaging

(71) Applicant: KARL STORZ SE & Co. KG, Tuttlingen (DE)

(72) Inventors: Lukas Buschle, Mühlheim (DE); Jasmin Ilg, Dürbheim (DE); Simon Haag, Rietheim-Weilheim (DE)

(73) Assignee: KARL STORZ SE & Co. KG, Tuttlingen (DE)

(21) Appl. No.: 19/120,638

(22) PCT Filed: Oct. 12, 2023
(86) PCT No.: PCT/EP2023/078377

§ 371 Date: Apr. 11, 2025

(30) Foreign Application Priority Data
Oct. 14, 2022 (DE) 10 2022 126 995.7

(51) Int. Cl. A61B 1/06; A61B 1/00; A61B 1/04; A61B 1/05

(52) U.S. Cl. CPC A61B 1/0638; A61B 1/000095; A61B 1/00057; A61B 1/00186; A61B 1/043; A61B 1/05

(57) ABSTRACT

The invention relates to a medical imaging device, comprising: an illumination unit having a light source, which is designed to provide illumination light for illuminating an object to be imaged; an image capturing unit, which is designed to take at least one calibration image of the object to be imaged and to take at least one object image of the object to be imaged; and an image correction unit. The image correction unit is designed to: determine depth information from the calibration image; determine a correction for the object image, wherein the correction comprises taking into account a location dependency, in particular a distance dependency, a light intensity of illumination light and/or a distance dependency of a light intensity of object light according to the depth information; and generate a corrected object image according to the correction. Also disclosed are an imaging method, program code, and corresponding computer product.

The invention relates to a medical imaging device, in particular an endoscope device, exoscope device and/or microscope device, to a method for medical imaging, to a program code for carrying out such a method, and to a computer program product comprising such a program code.

Imaging devices for performing fluorescence imaging are known from the prior art and can capture both fluorescence images and white light images. Suitable excitation light is used to specifically excite fluorescent dyes or, where possible, natively occurring fluorescent substances, and to detect emitted light and use it for imaging. In order to be able to show anatomical structures in the image region to a user at the same time, a white light image is often taken in parallel or sequentially. Using the white light image, the user can assess whether the anatomical structure is being imaged. Fluorescence images and white light images can also be superimposed, allowing anatomical information and fluorescence information to be perceived and analyzed by a user at the same time.

Imaging devices, such as endoscopic or exoscopic devices that produce multispectral or hyperspectral images, are also known from the prior art. Multispectral or hyperspectral images have a spectral dimension in addition to two spatial dimensions, such as a conventional image from a camera. The spectral dimension includes multiple spectral bands (wavelength bands). Multispectral and hyperspectral images differ substantially in the number and width of their spectral bands. Such systems can in principle also be suitable for taking fluorescence images.

Some imaging devices are known for producing such multispectral or hyperspectral images, especially in the context of medical applications. For example, DE 20 2014 010 558 U1 describes a device for capturing a hyperspectral image of an examination region of a body. The device includes an input lens for generating an image in an image plane and a slit-shaped aperture in the image plane for masking out a slit-shaped region of the image. The light passing through the aperture is spread out by a dispersive element and recorded by a camera sensor. This allows the camera sensor to record a plurality of spectra, each with an associated spatial coordinate, along the longitudinal direction of the slit-shaped aperture. The device described is further configured to record further spectra along the longitudinal direction of the slit-shaped aperture in a direction different from the longitudinal direction of the slit-shaped aperture. The method underlying this disclosure for generating multispectral or hyperspectral images is also known as the so-called pushbroom method.

In addition to the pushbroom method, there are other methods for generating multispectral or hyperspectral images. In the so-called whiskbroom method, the region under study or else the object is scanned point by point and a spectrum is obtained for each point. In contrast, the staring method involves taking multiple images with the same spatial coordinates. Different spectral filters and/or illumination sources are used from image to image to resolve spectral information. Furthermore, there are methods according to which a two-dimensional multi-color image is broken down into a plurality of individual spectral images using suitable optical elements, such as optical slicers, lenses and prisms, which images are simultaneously captured on different detectors or detector regions. This is sometimes referred to as the snapshot approach.

As described in DE 10 2020 105 458 A1, multispectral and hyperspectral imaging devices are particularly suitable as endoscopic imaging devices. In this context, multispectral and/or hyperspectral imaging is a fundamental field of application, for example for diagnostics and for assessing the success or quality of an intervention.

Multimodal imaging devices allow the capture of, optionally, white light images and/or multispectral images and/or fluorescence images and/or hyperspectral images. Examples of such imaging devices are multimodal endoscopes and multimodal exoscopes.

In general, in the imaging techniques mentioned, light of a specific spectrum is irradiated onto the object being observed and then reflected, absorbed, transmitted or emitted as a result of fluorescence excitation. Ultimately, light reaches an image sensor, optionally through one or more suitable observation filters. The image sensor captures image data which can be used to create a depiction for a user. There is no qualification as to which light interaction the detected light is due to.

Proceeding from the prior art, the object addressed by the invention is that of improving the interpretability of image data.

This object is achieved according to the invention by an imaging device, a method for medical imaging, a program code, and a computer program product as described herein and defined in the claims.

According to the invention, a medical imaging device, in particular an endoscope device, exoscope device and/or microscope device, comprises an illumination unit with at least one light source which is configured to provide illumination light for illuminating an object to be imaged, and an image capturing unit which is configured to capture at least one calibration image of the object to be imaged and to capture at least one object image of the object to be imaged. The imaging device also comprises an image correction unit. The image correction unit is configured to determine depth information from the calibration image. Furthermore, the image correction unit is configured to determine a correction for the object image, wherein the correction comprises taking into account a location dependency, in particular a distance dependency, of a light intensity of illumination light and/or a distance dependency of a light intensity of object light, in accordance with the depth information. In addition, the image correction unit is configured to generate a corrected object image in accordance with the correction.

Furthermore, the invention provides a method for medical imaging. In some embodiments, the method can be performed with a medical imaging device according to the invention. The method comprises providing illumination light for illuminating an object to be imaged. In addition, the method comprises capturing at least one calibration image of the object to be imaged. The method also comprises capturing at least one object image of the object to be imaged. The method further comprises determining depth information from the calibration image. Furthermore, the method comprises determining a correction for the object image, wherein the correction comprises taking into account a distance dependency of a light intensity of illumination light and/or a distance dependency of a light intensity of object light in accordance with the depth information. The method further comprises generating a corrected object image in accordance with the correction.

The features according to the invention make it possible to improve the interpretability of image data. The inventors have recognized that in conventional fluorescence imaging, multispectral imaging, or hyperspectral imaging, signal intensity varies with distance, which may lead to image data being misinterpreted. The aforementioned distance dependency may mean that reflectance values cannot be measured absolutely in multispectral imaging and/or hyperspectral imaging. The inventors have also recognized that the accuracy of the interpretation of image data can be impaired if fluorescence signals are attenuated by overlaid tissue and/or if it is not known how deep in the observed tissue the observed fluorescent dye is located or how great is the distance from an anatomical surface to the fluorescent dye. Furthermore, the inventors have identified as another problem with conventional multispectral imaging and/or hyperspectral imaging that for certain applications, in particular for the calculation of physiological parameters such as tissue oxygen saturation (StO2 parameter), simple assumptions have been made regarding the penetration depth of light, which can lead to incorrect measurement results. According to the invention, the effects mentioned can be compensated by determining depth information from a calibration image and using it to correct object images. The physical interaction of illumination light, remitted light and/or emitted light with the observed object can be taken into account, making the available information more accurately interpretable.

The imaging device can be configured as a microscope, macroscope and/or exoscope and/or comprise such. In some embodiments, the imaging device can be an endoscopic imaging device. It can comprise an endoscope and/or an endoscope system and/or be configured as such and/or form at least a part and preferably at least a major part and/or main component of an endoscope and/or an endoscope system. “At least a major part” can mean at least 55%, preferably at least 65%, more preferably at least 75%, particularly preferably at least 85% and most preferably at least 95%, in particular with reference to a volume and/or a mass of an object.

In some embodiments, the imaging device is configured to be insertable into a cavity for inspection and/or observation, for example into an artificial and/or natural cavity, such as into the interior of a body, into a body organ, into tissue or the like.

In particular if the imaging device is an exoscopic imaging device, it may be configured to acquire tissue parameters, images of wounds, images of body parts, etc. For example, the imaging device may be configured to image a surgical field.

The image capturing unit comprises in particular an image capturing sensor and/or at least one optical element, in particular a lens.

The image capturing sensor can be configured to detect light in both the visible and near-infrared range. A minimum detectable wavelength may, in some embodiments, be at most 500 nm, at most 450 nm, or even at most 400 nm. A maximum detectable wavelength can be at least 800 nm, at least 900 nm, or even at least 1000 nm in some embodiments. The image capturing sensor may, for example, comprise at least one white light image sensor and at least one near-infrared image sensor. In some embodiments, the imaging device comprises a white light camera and/or sensor system for white light imaging. The imaging device can be configured for white light imaging. The anatomical images can be captured using the white light camera and/or the sensors for white light image capture.

The image capturing unit can comprise a filter unit with optical observation filters. The filter unit can define multiple observation modes and/or fluorescence modes defined using different observation filters. For example, different edge filters can be used which absorb/block the respectively used spectrum of the corresponding luminous element used for excitation and at least substantially only transmit fluorescent light. In some embodiments, the observation filters may also be switchable between a multispectral mode and/or a hyperspectral mode and a fluorescence mode.

The imaging device and in particular the lens of the image capturing unit and/or the image capturing sensor can be configured for multispectral and/or hyperspectral imaging, in particular for capturing and/or generating multispectral and/or hyperspectral image data. Multispectral imaging or multispectral image data can refer in particular to such imaging in which at least two, in particular at least three, and in some cases at least five spectral bands can be acquired and/or are acquired independently of one another. Hyperspectral imaging or hyperspectral image data can refer in particular to imaging in which at least 20, at least 50 or even at least 100 spectral bands can be acquired and/or are acquired independently of one another. The imaging device may operate according to the pushbroom method, and/or the whiskbroom method, and/or according to the staring method, and/or according to a snapshot principle.

For some applications it can be advantageous to be able to use a high spectral resolution. Hyperspectral imaging is then recommended. This can be combined with white light imaging. This makes real-time observation possible via a white light image, even if the acquisition of spectrally resolved image data only occurs substantially in real time, i.e., for example, several seconds are needed to create a spectrally resolved image. For some applications it can be advantageous to generate spectral image data in real time. This includes, for example, the generation of a spectrally resolved image in less than a second or even multiple times per second. It can be useful to use multispectral imaging in this case. An optionally lower spectral resolution is then offset by a higher refresh rate. Depending on the application, it can be sufficient to consider only a few different spectral ranges and/or wavelengths, for example two or three or four or generally less than ten. In this case, additional white light imaging can optionally be omitted. Spectrally resolved image data that are acquired in real time or deliver several images per second can also be used for monitoring purposes, wherein it is not absolutely necessary to create a reproducible image for a user, but rather the image data can also be processed in the background.

The medical imaging device can have at least a proximal portion, a distal portion and/or an intermediate portion. The distal portion is in particular configured to be introduced into and/or located in a cavity to be examined in an operating state, for example during the diagnostic and/or therapeutic activity. The proximal portion is in particular configured to be arranged outside the cavity to be examined in an operating state, for example during the diagnostic and/or therapeutic activity. “Distal” should be understood to mean, in particular, facing a patient and/or facing away from a user during use. “Proximal” should be understood to mean, in particular, facing away from a patient and/or facing a user during use. In particular, proximal is the opposite of distal. The medical imaging device in particular has at least one, preferably flexible, shaft. The shaft can be an elongated object. Furthermore, the shaft can at least partially and preferably, at least to a large extent, form the distal portion. An “elongated object” is to be understood in particular as an object whose main extension is at least a factor of five, preferably at least a factor of ten and particularly preferably at least a factor of twenty larger than a largest extension of the object perpendicular to its main extension, i.e. in particular a diameter of the object. A “main extension” of an object should be understood in particular as its longest extension along its main extension direction. A “main extension direction” of a component is to be understood in particular as a direction which runs parallel to a longest edge of a smallest imaginary cuboid which only just completely encloses the component.

The image capturing unit can be arranged at least partially and preferably at least to a large extent in the region of the proximal portion and/or can form it. In other embodiments, the image capturing unit may be arranged at least partially and preferably at least to a large extent in the distal portion and/or can form it. Furthermore, the image capturing unit can be arranged at least partially distributed over the proximal portion and the distal portion. The image capturing sensor system comprises in particular at least one image sensor. Furthermore, the image capturing sensor system can also have at least two and preferably several image sensors, which can be arranged one behind the other. Furthermore, the two and preferably several image capturing sensors can have spectral acquisition sensitivities different from one another so that, for example, a first sensor in a red spectral range, a second sensor in a blue spectral range and a third sensor in a green spectral range is particularly sensitive or comparatively more sensitive than the other sensors. The image sensor can be configured as a CCD sensor and/or a CMOS sensor.

An optics of the image capturing unit can comprise suitable optical elements, such as lenses, mirrors, gratings, prisms, optical fibers, etc. The optics can be configured to guide object light coming from an imaged object to the image capturing sensor, for example to focus and/or project it.

The image capturing unit is particularly designed to capture spatially and spectrally resolved image data. The image capturing unit can in particular be configured to generate at least two-dimensional spatial image data. The image capturing unit can be spatially resolving in such a way that it provides a resolution of at least 100 pixels, preferably of at least 200 pixels, preferably of at least 300 pixels and advantageously of at least 400 pixels in at least two different spatial directions. The image data are preferably at least three-dimensional, wherein at least two dimensions are spatial dimensions and/or wherein at least one dimension is a spectral dimension. From the image data, multiple spatially resolved images of the image region can be obtained, each of which is assigned to different spectral bands. The spatial and spectral information of the image data can be such that an associated spectrum can be obtained for a plurality of spatial pixels.

In some embodiments, the image capturing unit is configured to generate continuously updated image data. The image capturing unit can, for example, be configured to generate the image data substantially in real time, which can comprise, for example, generating updated image data at least every 30 seconds, in some cases at least every 20 seconds, and in some cases even at least every 10 seconds or at least every 5 seconds. Preferably, the image capturing unit is configured to generate at least the anatomical images and the fluorescence images as well as the depiction based thereon in real time, for example with a frame rate of at least 5 fps, at least 10 fps, at least 20 fps or even at least 30 fps.

The illumination unit can be multimodal and can comprise a plurality of luminous elements which are optionally activatable independently of one another and which are configured to emit light according to different emission spectra in order to provide the illumination light.

The image capturing unit can be operable in a calibration mode and in at least one imaging mode. In the calibration mode, at least one calibration image can be captured. In the imaging mode, at least one object image can be captured. The object image can be a white light image, a fluorescence image, a multispectral image and/or a hyperspectral image. An image portion of the calibration image can correspond to an image portion of the object image, and/or at least overlap therewith. In particular, the calibration image can define an image portion that is at least partially contained in an image portion of the object image. The correction can be carried out in particular for regions of the object image that are also depicted in the calibration image. The image correction unit can be configured to compare image portions of the calibration image and the object image in order to identify image regions that can be corrected. This allows a correction to be made even in cases where the calibration image and the object image do not coincide.

The image capturing unit can be configured to capture multiple calibration images with different image capture parameters and/or illumination parameters. The depth information can be based on multiple calibration images, especially those taken with different parameters.

The image capturing unit can be configured to capture stereo images. For example, the image capturing unit can comprise at least one pair of image sensors so that stereo pairs of images can be captured.

For the purposes of this disclosure, object light generally refers to light emanating from an object being observed. As mentioned, this can be remitted light or emitted light, depending on the nature of the object and/or the type of imaging. In some embodiments, the correction comprises assigning spatial coordinates x, y, z to captured pixels according to the depth information. Based on this, the correction for a specific pixel can be based on a function ƒ (x,y,z) which depends on the spatial coordinates x, y, z of the specific pixel. If the correction takes into account a distance dependency, the function can be based on the sum and/or the square root of this sum x²+y²+z² and/or the square root of this sum, i.e., sqrt (x²+y²+z²). The correction can be performed point by point and/or image region by image region and/or image by image.

The corrected object image can be corrected point by point and/or image region by image region and/or image by image. The corrected object image is based in particular on the object image and the calibration image.

The above-mentioned method steps can be carried out in the order in which the method steps are named. However, it is understood that a different sequence is also possible according to the invention, and the list of method steps does not necessarily define a predetermined sequence.

A comprehensive correction can be carried out in particular if the depth information comprises at least one depth map. A depth map is understood to mean, in particular, spatially resolved depth information which assigns at least one depth value, which is defined, for example, by a coordinate z, to a series of pixels, in particular each pixel of the calibration image which can be defined by coordinates x and y. In other words, a topography of the object to be imaged can be derived from the calibration image in order to obtain the depth information. This depth information can be specific to the image capture parameters and/or illumination parameters used. The depth information may include multiple depth maps relating to different wavelength ranges and/or tissue types and/or anatomical structures. Different depth maps can also be determined alternatively or additionally from multiple calibration images. In particular, at least one calibration image can be taken with specific parameters in order to obtain a specific depth map.

An image capture of the calibration image may include a detection of remitted light. This allows depth information to be obtained in a simple way by taking advantage of the fact that light in certain spectral ranges has a very low penetration depth into tissue. In this case, the calibration image is preferably captured using stereo imaging or 3D imaging. The calibration image can be a 3D image. A depth map can then be obtained using a stereo reconstruction algorithm. For example, a semi-global matching algorithm can be used for this purpose, as described in the article “Accurate and efficient stereo processing by semi-global matching and mutual information” by Hirschmüller, 2005, IEEE Conference on Computer Vision and Pattern Recognition. pp. 807-814). Alternatively, a 3D image or a topographical surface can be calculated from two-dimensional image data. For example, an artificial intelligence algorithm can be trained in advance with suitable 2D and 3D image data. The capture and/or evaluation of 3D images may include a calibration of the image capturing unit aimed at determining distortion parameters and/or a relative spatial position of image sensors. Light used to capture such calibration images is substantially remitted directly from the surface of the object to be imaged, which is why the calibration image primarily or at least substantially exclusively comprises image information relating to the surface of the object to be imaged. A depth map can be determined, for example, from a white light calibration image. White light typically has a very shallow penetration depth into tissue, so that remitted light approximately originates from an anatomical surface. Alternatively or additionally, a depth map can be obtained from a single-color calibration image or a false-color calibration image using illumination light that lies only in one or more subranges of visible light. For example, it is possible to use only blue light, green light, yellow light and/or red light to obtain depth information based on remitted light. A mixture of certain colors and/or spectral ranges that deviates from white light can also be used. Preferably, wavelength ranges are used for which light has the lowest possible penetration depth into the object to be imaged, so that it is ensured that the remitted light originates from the surface of the object. Alternatively or additionally, different calibration images can be taken for different wavelength ranges, and depth maps can be calculated for each. These can then be used for the correction in different wavelengths, which allows wavelength-dependent penetration depths in particular to be taken into account.

In some embodiments, an image capture of the calibration image may include detecting fluorescent light. This can provide a basis for a comprehensive and accurate evaluation of fluorescence images. Preferably, at least one 3D fluorescence image is captured. As described above, light with a suitable wavelength can be irradiated as excitation light and light emitted by the object to be imaged can be detected with a suitable observation filter. Depth information, in particular a depth map, can be obtained by stereo reconstruction. Alternatively, analogous to the case described above, a 2D image can be used as a basis, and depth information can be obtained using an artificial intelligence algorithm. In particular, a depth map can be obtained from the calibration image, which refers to regions of the object to be imaged that lie within the object and/or below a surface of the object. In particular, the depth map can be derived from stained regions covered by unstained tissue. Fluorescent light then reaches the image sensor and/or a lens of the image capturing unit from a position that is further away from the image sensor and/or the lens of the image capturing unit than a surface of the object to be imaged.

A comprehensive correction that allows a correct interpretation of image data in different imaging modes can be achieved in particular if the illumination unit is configured to be operable in different illumination modes in which illumination light can be provided in different spectral ranges, and the image capturing unit is configured to acquire a plurality of calibration images whose image capture is based on different illumination modes of the illumination unit. For example, a first illumination mode can be used to obtain a calibration image based on remitted light, and a second illumination mode can be used to obtain a calibration image based on emitted light, in particular fluorescent light. From this, at least one first depth map and at least one second depth map can be obtained, wherein the first depth map relates to a surface of the object to be imaged and wherein the second depth map relates to regions of the object to be imaged that are stained by means of at least one fluorescent dye and are located below the surface of the object to be imaged.

The image capturing unit can be configured to acquire multiple calibration images in different spectral ranges simultaneously and/or sequentially, in particular using different optical filters. For example, multiple calibration images can be based on different spectral ranges in multispectral imaging and/or hyperspectral imaging.

In some embodiments, the correction comprises a distance correction based on an inverse of a power of a length of a light path, in particular a length of a light path between the image capturing unit, in particular an image sensor and/or a lens of the image capturing unit, and the object to be imaged, and/or a light path within the object to be imaged. The distance correction can be based, for example, on an inverse square law. In this case in particular, the illumination unit can be considered approximately as a point light source. Deviations from a point light source can be taken into account by using an exponent other than 2.

Furthermore, the correction can comprise an absorption correction based on an attenuation, in particular exponential attenuation, of light along a light path having a length—in particular, a length of a light path within the object to be imaged. Alternatively or additionally, the absorption correction may take into account an attenuation of illumination light and/or an attenuation of object light.

Generally speaking, a distance correction for fluorescence imaging, wherein light with a wavelength λ₀ is irradiated and light with a wavelength λ₁ is emitted, may be based on the following attenuation, due to a distance from a lens of the image capturing unit to the object to be imaged, and also a positioning of a region stained with fluorescent dye in the object to be imaged:

$${I}_{detected} = I ( {Λ}_{0} ) / {( {d}_{0} + {d}_{1} )}^{α} \cdot exp ( - μ ( {Λ}_{0} ) \cdot {d}_{1} ) \cdot exp ( - μ ( {Λ}_{1} ) \cdot {d}_{1} ) \cdot R$$

where I_{detected} denotes the detected light intensity, I(λ₀) denotes the intensity of the incident light with wavelength λ₀, d₀ is a distance between the lens and the surface of the object to be imaged, d₁ is a distance between the surface of the object to be imaged and the region in the object to be imaged that is stained with fluorescent dye, α is an exponent that defines the inverse law and can be chosen, for example, to be 2, so as to calculate according to the inverse square law, μ(λ₀) is an attenuation factor for the attenuation of light of the wavelength λ₀ when it passes through the object to be imaged, and μ(λ₁) is an attenuation factor for the attenuation of light of the wavelength λ₁ when it passes through the object to be imaged.

A particularly high degree of accuracy can be achieved in particular if the image correction unit is configured to determine spatial and/or spectral properties, in particular inhomogeneities, of the illumination unit from the at least one calibration image and to take the determined spatial and/or spectral properties into account in the correction. In other words, the capturing of calibration images can be used to calibrate the illumination device or to take real properties of the illumination device into account for the calculation of the corrected object image.

In some embodiments, it can be provided that in the corrected object image at least one image region is enhanced and/or attenuated according to the correction relative to at least one other image region with respect to at least one parameter, such as a hue, a brightness and/or a color saturation. This allows a corrected object image to be created that is intuitively understandable for a user. In general, this can be used to compensate for differences in intensity that are due to different lighting situations but not to differences in the tissue being imaged. The corrected object image can then, for example, be calculated in such a way that the intensity differences are not recognizable. For the user, for example, similar regions can then be recognized as such, even if they are not imaged in the same way. For example, a fluorescing region that is partially or completely covered by unstained tissue can be displayed as if it were not covered. Alternatively, distance information can be transmitted to the user by using false colors. For example, a fluorescing region covered by unstained tissue can be displayed with increased brightness so that it is clearly visible, but a color can be changed according to a distance of the fluorescing region from the surface of the object to be imaged, so that the user can see whether and how far the fluorescing region is located within the object to be imaged. This can, for example, assist the user in surgical preparation.

In some embodiments, the medical imaging device comprises an illumination device comprising the illumination unit. The illumination device can comprise an optical interface for optically connecting an imaging device. The illumination unit can be designed to provide illumination light to the optical interface. The illumination unit can be multimodal and can comprise a plurality of luminous elements which are optionally activatable independently of one another and which are configured to emit light according to different emission spectra in order to provide the illumination light. The illumination unit may be operable in at least one multispectral mode in which a first group of the luminous elements is at least temporarily activated and in which the illumination unit provides illumination light for multispectral imaging. The illumination unit may further be operable in at least one fluorescence mode in which a second group of the luminous elements is at least temporarily activated and in which the illumination unit provides illumination light for fluorescence imaging. The luminous elements can comprise at least one luminous element which is included in both the first group and the second group.

In addition, a method for generating illumination light for an imaging device by means of an illumination device is provided. The illumination device comprises an optical interface for the optical connection of an imaging unit, and an illumination unit that is designed to provide illumination light to the optical interface, wherein the illumination unit comprises a plurality of luminous elements which are optionally activatable independently of one another and which are configured to emit light according to different emission spectra in order to provide the illumination light. The method comprises the step of at least temporarily activating a first group of the luminous elements to provide illumination light for multispectral imaging and the step of at least temporarily activating a second group of the luminous elements to provide illumination light for fluorescence imaging. At least one of the luminous elements is activated at least temporarily both when the first group of luminous elements is activated at least temporarily and when the second group of luminous elements is activated at least temporarily.

The optical interface can be either detachable or connectable. In addition, the optical interface can be combined with a mechanical interface so that an optical connection is automatically established, for example, when the imaging unit is mechanically coupled.

The luminous elements can comprise single-color LEDs (light-emitting diodes) and/or laser diodes. Furthermore, at least one of the luminous elements can be a white light LED or other white light source. In some embodiments, the illumination unit comprises at least one blue luminous element, at least one red luminous element, at least one dark red luminous element and at least one near-IR luminous element (near-infrared luminous element), in particular LEDs or laser diodes in each case. In addition, the illumination unit can comprise at least one white light LED or other white light source.

The first group can comprise at least two luminous elements that emit spectrally differently. A high degree of efficiency in multispectral imaging can be achieved if the multispectral mode comprises different states in which a specific luminous element or a specific type of luminous element is activated at least temporarily. This allows targeted illumination in a specific spectral range, allowing different spectral images to be captured. Different luminous elements activated in different states can serve as different support points for multispectral imaging. At least one of these support points can be selected such that it is adapted to characteristic points of absorption spectra of physiologically relevant components, for example to an isosbestic point of the hemoglobin oxygenation curve. Multispectral imaging can additionally include the use of appropriate observation filters.

Furthermore, the second group can comprise at least two luminous elements that emit spectrally differently. The fluorescence mode can comprise different submodes and/or states in which a specific luminous element or a specific luminous element type is activated at least temporarily. This allows targeted excitation in a certain spectral range, so that fluorescence imaging can be carried out, for example, for a specifically selected dye. In other words, the at least one luminous element contained in both the first group and the second group can be used for both the multispectral mode and the fluorescence mode.

In some embodiments, the first group comprises only some but not all of the luminous elements. Alternatively or additionally, in some embodiments, the second group comprises only some but not all of the luminous elements. In the multispectral mode only luminous elements of the first group, in particular, are activated at least temporarily, whereas luminous elements that do not belong to the first group are deactivated. In the fluorescence mode only luminous elements of the second group, in particular, are activated at least temporarily, whereas luminous elements that do not belong to the second group are deactivated. In general, it shall be understood that the luminous elements can comprise different luminous element types and that exactly one luminous element of each of the different luminous element types, in particular, can be present. It shall be understood that mixed operating modes can also occur according to the invention, in which said modes are used sequentially. For example, multispectral imaging and fluorescence imaging can be performed sequentially.

Synergy with regard to the use of a luminous element for different modes and associated efficiency gains can be achieved in particular if at least one luminous element contained in both the first group and the second group emits light in the red spectral range, in particular in a spectral range between 600 nm and 680 nm, for example between 610 nm and 650 nm or between 620 and 660 nm or between 630 and 670 nm. The spectral range can be narrowband and cover the 660 nm wavelength. “Narrowband” may include a spectral width of at most 80 nm, in particular at most 40 nm or even at most 20 nm. This at least one luminous element can be configured to excite dyes absorbing in the red spectral range and to contribute to the illumination in the red spectral range for multispectral imaging.

In some embodiments, the illumination unit may be operable in at least one white light mode in which the illumination unit provides illumination light for white light imaging. The illumination light for white light imaging can be broadband white light. Alternatively, the illumination light for white light imaging can comprise a plurality of narrow wavelength bands that are separated from one another, for example a blue, a red and a dark red band. “Dark red” is to be understood as having a “longer wavelength than red” and refers to the spectral position, but not to light intensity. The illumination light for white light imaging can be a mixture of light from different luminous elements.

In the white light mode, a third group of luminous elements may be activated at least temporarily to provide the illumination light for white light imaging. The luminous elements can comprise at least one luminous element which is contained both in the first group and/or second group and in the third group. In some cases, the third group may comprise only some but not all of the luminous elements. In the white light mode only luminous elements of the third group, in particular, are activated at least temporarily, whereas luminous elements that do not belong to the third group are deactivated. In other words, the illumination unit may comprise luminous elements that serve one, two or all three of the aforementioned illumination modes. This allows for a plurality of luminous elements to be used multiple times.

At least one luminous element contained both in the first group and/or second group and in the third group can emit light in the red spectral range, in particular in a spectral range between 600 nm and 680 nm, for example between 610 nm and 650 nm or between 620 and 660 nm or between 630 and 670 nm. The advantages of using luminous elements together are particularly evident when at least one red luminous element can be used for all three modes.

At least one luminous element contained both in the first group and/or second group and in the third group can emit light in the blue spectral range, in particular in a spectral range between 440 and 480 nm. At least one blue luminous element can be conveniently used both in the fluorescence mode and in the white light mode.

Generally speaking, the luminous elements, as mentioned, can comprise at least one, in particular blue, luminous element which emits light in a spectral range between 440 and 480 nm. Moreover, the luminous elements, as mentioned, can comprise at least one, in particular red, luminous element which emits light in a spectral range between 600 and 680 nm, for example between 610 nm and 650 nm or between 620 and 660 nm or between 630 and 670 nm. Alternatively or additionally, the luminous elements can comprise at least one, in particular dark red, luminous element which emits light in a spectral range between 750 and 790 nm. Alternatively or additionally, luminous elements can comprise at least one, in particular near-IR emitting, luminous element that emits light in a spectral range between 920 and 960 nm. In addition, the luminous elements can include a white light luminous element. A compact and versatile illumination unit can be provided in particular if at least one luminous element of each of the above-mentioned luminous element types is present. For example, in fluorescence mode, the blue and the red luminous element can be used, and in the case of suitable dyes, the dark red luminous element may also be used. In multispectral mode, the dark red and near-IR emitting luminous elements can be used. In white light mode, the white light luminous element can be used. In white light mode, it can be supplemented by the blue luminous element and, if necessary, the red luminous element. This makes it possible to supplement wavelength ranges by means of colored luminous elements in which the white luminous element provides a reduced intensity, for example due to its construction but in particular due to filters and optical elements of the illumination unit. In addition, the colored luminous elements can be used to adjust a color temperature for white light imaging.

In some embodiments, the second group comprises a single luminous element and/or a single type of luminous elements. For example, a white light luminous element, a red luminous element and an IR-emitting luminous element can be provided, with particular reference being made to the above values as regards possible spectral ranges. The first group can then, for example, comprise the red and the IR-emitting luminous element. The second group can comprise the IR-emitting luminous element, in particular as the only luminous element or as the only type of luminous element.

A favorable arrangement of luminous elements is made possible in particular if the illumination unit comprises at least one crossed beam splitter, by means of which light can be deflected from opposite input sides to an output side, wherein at least one of the luminous elements is arranged on the opposite input sides of the crossed beam splitter. In some embodiments, two or more crossed beam splitters may be provided that are arranged optically one behind the other. The at least one crossed beam splitter can comprise two beam splitter elements, the transmittance of which is adapted to the respectively associated luminous element. The beam splitter elements each comprise, in particular, a notch filter so that they reflect in a narrow spectral band but otherwise transmit. The spectral position and/or width of the corresponding notch can be adapted to the spectral range of the respectively associated luminous element, so that its light is redirected, but light from other luminous elements is at least largely transmitted.

In some embodiments, the luminous elements can comprise at least four narrowband emitting single-color luminous elements, each with different spectral ranges, and at least one broadband emitting white light luminous element. In this regard, reference is also made to what has been said above in connection with colored luminous elements.

A wide range of functions in combination with a compact design and the utilization of synergy effects when using luminous elements can be achieved in particular if the illumination unit is operable in at least one hyperspectral mode in which a plurality of luminous elements is activated, the emission spectra of which together cover at least a spectral range from 450 nm to 850 nm, and in which the illumination unit provides illumination light for hyperspectral imaging. This may in particular include all of the luminous elements.

It shall be understood that, in particular when using laser diodes, suitable polarization filters can be used for the optical filters mentioned herein. Furthermore, in particular when using laser diodes, at least one crossed beam splitter can be used, the beam splitter elements of which are provided with polarization filters. Selective transmittance can then be achieved by combining different polarizations.

The invention also relates to program code which, when executed in a processor, is designed to carry out of a method according to the invention.

Furthermore, the invention relates to program code comprising a computer-readable medium on which the program code according to the invention is stored.

The devices and systems according to the invention and the methods according to the invention should not be limited to the application and embodiment described above. In particular, they can have a number of individual elements, components and units as well as method steps, which differ from a number mentioned herein, in order to fulfill a function described herein. In addition, for the ranges of values specified in this disclosure, values within the stated limits shall also be deemed to be disclosed and to be usable in any manner.

It is in particular pointed out that all features and properties described with regard to a device, but also procedures, can be analogously transferred to methods and can be used within the meaning of the invention and are considered to be co-disclosed. The same applies in the opposite direction. This means that structural features mentioned in relation to methods, i.e., features relating to the device, can also be taken into account, claimed and also counted as part of the disclosure within the scope of the device claims.

The present invention is described below by way of example with reference to the accompanying figures. The drawings, the description, and the claims contain numerous features in combination. A person skilled in the art will also, expediently, consider the features individually and use them in combination as appropriate in the context of the claims.

If there is more than one example of a particular object, only one of them may be provided with a reference sign in the figures and in the description. The description of this example can be transferred accordingly to the other examples of the object. If objects are named using numerical words, such as first, second, third object, etc., these are used to name and/or assign objects. Accordingly, for example, a first object and a third object may be included, but not a second object. However, a number and/or sequence of objects could also be derived using numerical words.

In the drawings:

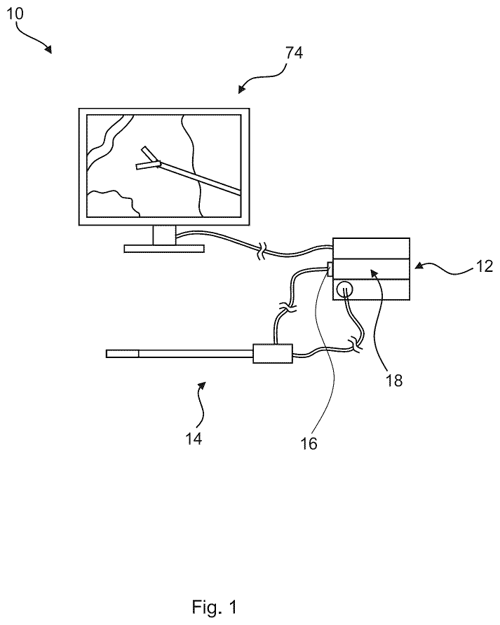

FIG. 1 is a schematic representation of an imaging device having an illumination device;

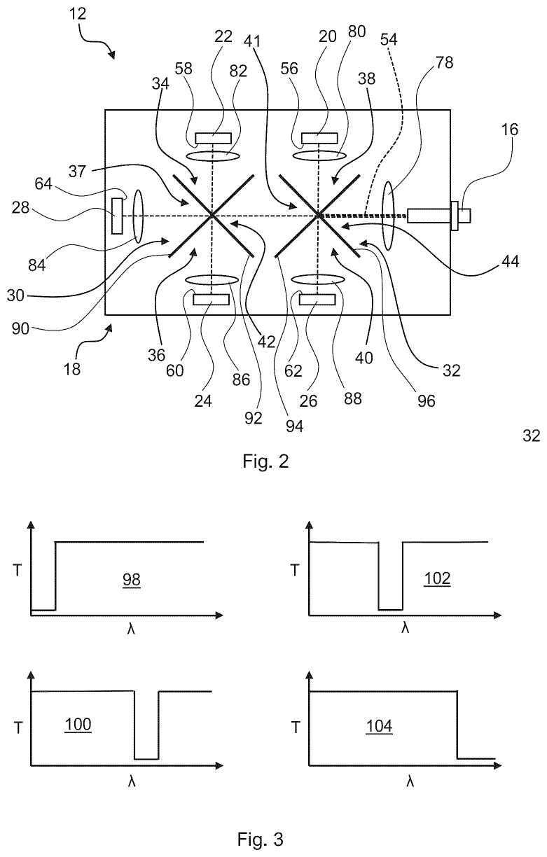

FIG. 2 is a schematic representation of the illumination device;

FIG. 3 is schematic transmission curves of beam splitter elements of the illumination device;

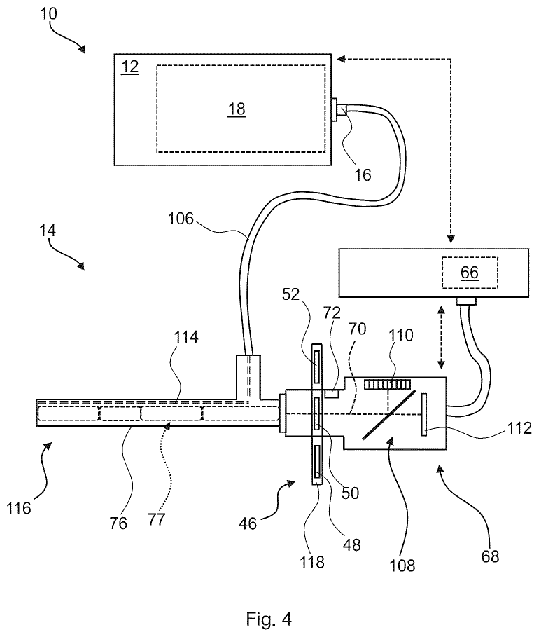

FIG. 4 is a schematic representation of the imaging device;

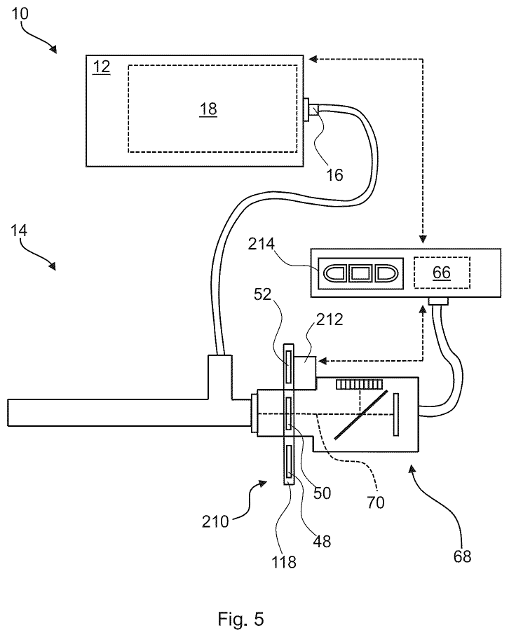

FIG. 5 is a schematic representation of another embodiment of the imaging device;

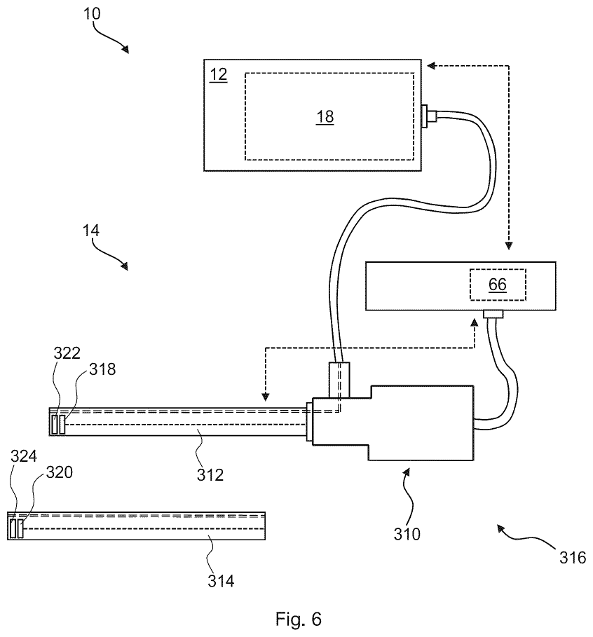

FIG. 6 is a schematic representation of yet another embodiment of the imaging device;

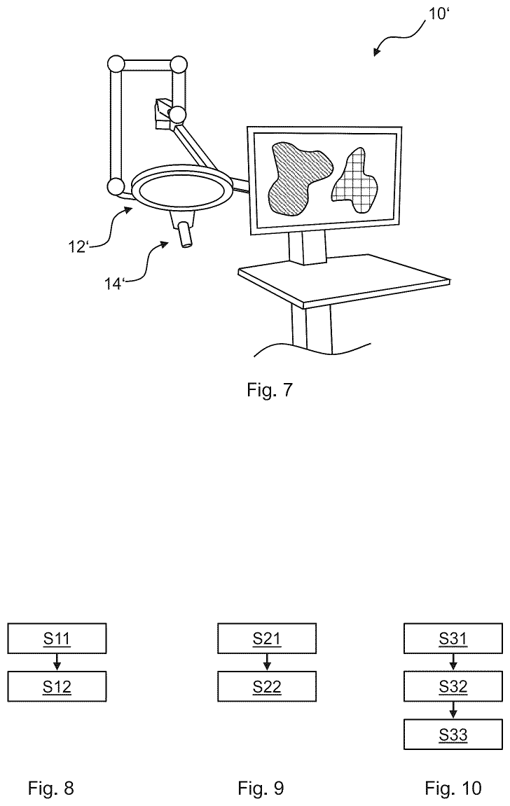

FIG. 7 is a schematic perspective representation of another embodiment of the imaging device;

FIG. 8 is a schematic flow chart of a method for generating illumination light for an imaging unit by means of an illumination device;

FIG. 9 is a schematic flow chart of a method for operating an imaging device;

FIG. 10 is a schematic flow chart of a method for operating an imaging device;

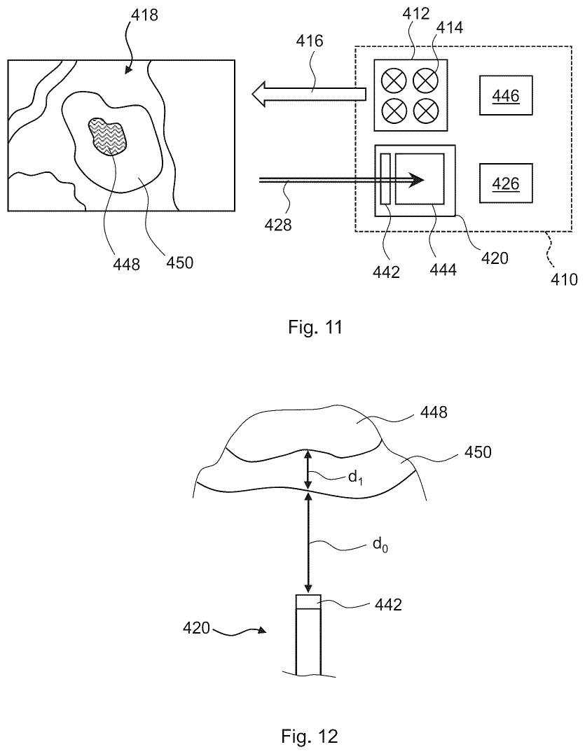

FIG. 11 is a schematic representation of a medical imaging device;

FIG. 12 is a schematic representation of an imaging situation;

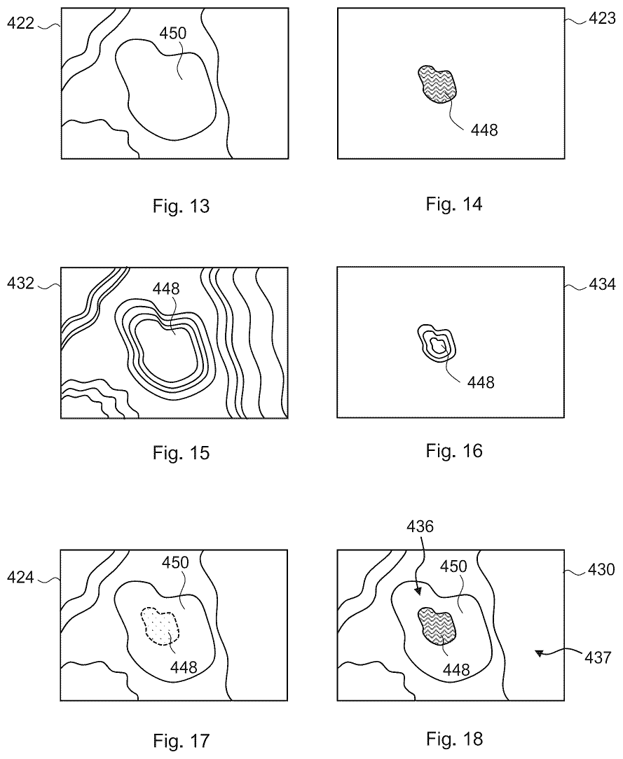

FIG. 13 is a schematic representation of a first calibration image;

FIG. 14 is a schematic representation of a second calibration image;

FIG. 15 is a schematic representation of a first depth map;

FIG. 16 is a schematic representation of a second depth map;

FIG. 17 is a schematic representation of an object image;

FIG. 18 is a schematic representation of a corrected object image;

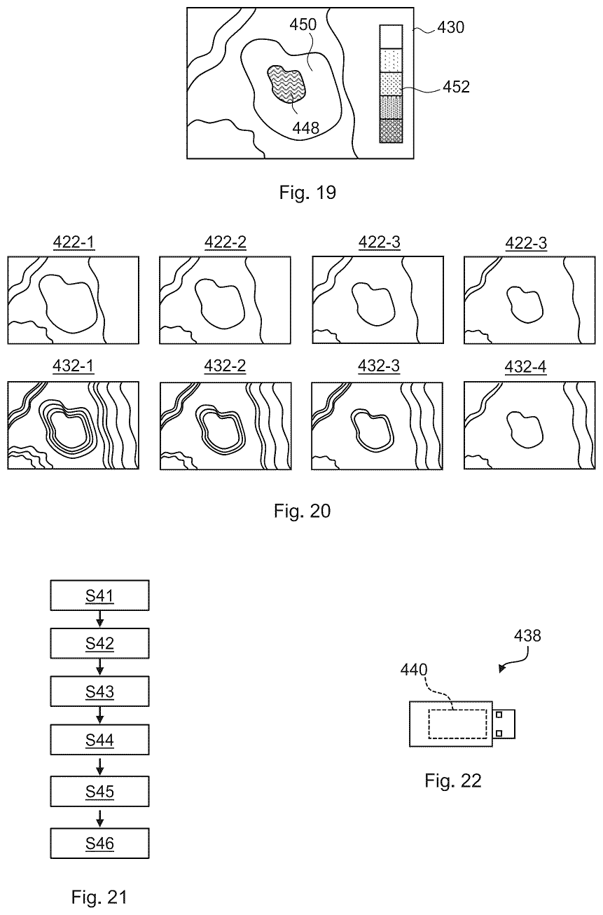

FIG. 19 is a schematic representation of a further, corrected object image;

FIG. 20 is a schematic representation of multiple calibration images and associated depth maps;

FIG. 21 is a schematic flow chart of a method for medical imaging; and

FIG. 22 is a schematic representation of a computer program product.

FIG. 1 shows a schematic representation of an imaging device 10. In the instance shown by way of example, the imaging device 10 is an endoscopic imaging device, specifically an endoscope device. Alternatively, the imaging device 10 could be an exoscopic, a microscopic or a macroscopic imaging device. The imaging device 10 is shown by way of example as a medical imaging device. The imaging device 10 is provided, for example, for examining a cavity.

The imaging device 10 comprises a medical imaging unit 14. In the shown case, this is an endoscope.

The imaging device 10 further comprises an illumination device 12 having an optical interface 16, and an illumination unit 18. The imaging unit 14 can be optically connected to the optical interface 16. The optical interface 16 can be part of an optical-mechanical interface that can be optionally detachable and connectable. The illumination apparatus 14 can optionally be decoupled from the illumination device 12. The illumination unit 18 is designed to provide illumination light to the optical interface 16. When imaging by means of the imaging unit 14, the illumination unit 18 can accordingly provide the required illumination light, which is guided to the illumination apparatus 14 and from there coupled out onto an object to be imaged, such as a situs.

In the illustrated case, the imaging device 10 further comprises a display unit on which images can be displayed that are based on image data acquired by means of the imaging unit 14. These can be video images, still images, overlays of different images, partial images, image sequences, etc.

The imaging device 10 is multimodal. By way of example, the imaging device can be operated in three basic modes: a multispectral mode, a fluorescence mode and a white light mode. It can further be provided for the imaging device 10 to be operable in a hyperspectral mode in addition or as an alternative to the multispectral mode.

The illumination device 12 is multimodal. The illumination device 12 is operable in different illumination modes in which it provides light for different imaging modes. Here, the illumination device 12 is operable in three basic modes, namely a multispectral mode, a fluorescence mode and a white light mode. The imaging unit 14 is also operable in different operating modes, specifically also in at least a multispectral mode, a fluorescence mode and a white light mode. In the corresponding operating mode of the imaging device 10, the modes of the illumination device 12 are matched to one another.

FIG. 2 shows a schematic representation of the illumination device 12. The illumination unit 18 comprises a plurality of independently activatable luminous elements 20, 22, 24, 26, 28. They are configured to emit light according to different emission spectra in order to provide illumination light, i.e., the relevant emission spectrum differs from luminous element to luminous element.

The luminous elements 20, 22, 24, 26, 28 are designed as LEDs, for example. Specifically, a first luminous element 20 is configured as a red LED, a second luminous element 22 as a dark red LED, a third luminous element 24 as a blue LED and a fourth luminous element 26 as a near-IR LED. The colored luminous elements 20, 22, 24, 26 each emit in a narrow band, for example with an emission peak for example at wavelengths 660 nm (first luminous element 20), 770 nm (second luminous element 22), 460 nm (third luminous element 24) and 940 nm (fourth luminous element 26).

A fifth luminous element 28 is further provided, which in the present case is a white light luminous element, for example a white light LED. The fifth luminous element 28 emits, for example, in a spectral range of about 400 to 700 nm. In other embodiments, laser diodes can also be used, in particular as colored luminous elements.

Depending on the illumination mode, some of the luminous elements 20, 22, 24, 26, 28 are activated at least temporarily, whereas other luminous elements 20, 22, 24, 26, 28 may not be used in the illumination mode in question.

In the present case, a first group comprises the first luminous element 20 and the fourth luminous element 26. The first group may additionally comprise luminous element 22 and/or luminous element 24. The first group is used for multispectral imaging, with the contained luminous elements 20, 26 and, if applicable, 22 and 24 each serving as support points. In multispectral mode, for example, the first luminous element 20 is initially used for illumination and an image is taken. Then, the fourth luminous element 26 is used for illumination and an image is taken. The images are based on remission, i.e., the light scattered back from the object to be imaged is considered. The two different support points can be used to obtain spectral information about the object to be imaged. For example, certain tissue types, a perfusion state, a tissue condition or the like can be assessed in this way.

Furthermore, a second group comprises the first luminous element 20, the second luminous element 22 and the third luminous element 24. The second group is used for illumination in fluorescence imaging. For example, objects that have been colored with appropriately selected dyes can be viewed. Also, different dyes can be introduced into different types of tissue or the like, which are viewed simultaneously. By specifically exciting a particular dye, it is excited to fluoresce. The fluorescent light is then imaged. The first luminous element 20 is suitable, for example, for exciting the dye cyanine 5.5 (Cy 5.5). The second luminous element 22 is suitable for exciting the dye indocyanine green (ICG). The third luminous element 24 is suitable for exciting the dye fluorescein.

Furthermore, a third group comprises the fifth luminous element 28. In the present embodiment, the third group moreover comprises the first luminous element 20 and the third luminous element 24. The third group is used to provide illumination light for white light imaging. For this purpose, white light from the fifth luminous element 28 can be mixed with light from certain colored luminous elements, whereby spectral losses can be compensated and/or a color temperature can be specifically adjusted.

It can be seen that some of the luminous elements 20, 22, 24, 26, 28 are assigned to a plurality of groups, for example the first luminous element 20 to all three groups and the third luminous element 24 and possibly also the second luminous element 22 to the second group and third group.

Alternatively or additionally, it may also be provided for some or all of the luminous elements 20, 22, 24, 26, 28 to be used in a hyperspectral mode. A broad spectrum of excitation is then generated. In combination with a suitable hyperspectral detector, spectral information about the object to be imaged can then be captured across the entire visible and near-IR spectrum. For this purpose, the imaging unit 14 may comprise a pushbroom arrangement as a hyperspectral detector. In other embodiments, a whiskbroom arrangement, a staring arrangement and/or a snapshot arrangement is used. The imaging unit 14 can be a hyperspectral imaging unit. Regarding different methods of hyperspectral imaging and components required for this, reference is made to the article “Review of spectral imaging technology in biomedical engineering: achievements and challenges” by Quingli Li et al., published in Journal of Biomedical Optics 18(10), 100901, October 2013, and to the article “Medical hyperspectral imaging: a review” by Guolan Lu and Baowei Fei, published in Journal of Biomedical Optics 19(1), 010901, January 2014.

The illumination unit 18 comprises two crossed beam splitters 30, 32. They each comprise an output side 42, 44, an input side 37, 41 opposite the output side 42, 44 and two input sides 34, 36, 38, 40 opposite one another. All input sides 34, 36, 37, 38, 40, 41 lead incident light to the corresponding output side 42, 44. The output side 42 of a first crossed beam splitter 30 faces an input side 41 of the second crossed beam splitter 32. The output side 44 of the second crossed beam splitter 32 faces the optical interface 16. The two crossed beam splitters 30, 32 are preferably arranged coaxially to one another and/or to the optical interface.

The illumination unit 18 can comprise suitable optical elements such as lenses and/or mirrors (not shown). A plurality of lenses 78, 80, 82, 84, 86, 88 is shown by way of example in FIG. 2. A lens 78 is assigned, for example, to the optical interface 16 and couples light coming from the output side 44 of the second crossed beam splitter 32 into the optical interface 16. Furthermore, each of the luminous elements 20, 22, 24, 26, 28 can be assigned a lens 80, 82, 84, 86, 88. A particularly high degree of compactness can be achieved in particular if the luminous elements 20, 22, 24, 26, 28 are each arranged without an intermediate mirror on input sides 34, 36, 37, 38, 40 of the at least one crossed beam splitter 30, 32. The luminous elements 20, 22, 24, 26, 28 can then be moved very close to the at least one crossed beam splitter 30, 32.

The crossed beam splitters 30, 32 each comprise two beam splitter elements 90, 92, 94, 96. They can basically be partially transmitting, so that light from all input sides 34, 36, 37, 38, 40, 41 is redirected to the relevant output side 42, 44. In the present embodiment, the beam splitter elements 90, 92, 94, 96 are selectively light-transmissive. This is illustrated with further reference to FIG. 3. The beam splitter elements 90, 92, 94, 96 can be filters that reflect only in a defined region, but otherwise have high transmission. FIG. 3 shows transmission curves 98, 100, 102, 104 of the beam splitter elements 90, 92, 94, 96 of the two crossed beam splitters 30, 32. Each of the colored luminous elements 20, 22, 24, 26 or each of the opposite input sides 34, 36, 38, 40 is assigned one of the beam splitter elements 90, 92, 94, 96. The beam splitter elements 90, 92, 94, 96 are selected such as to each reflect in the wavelength range in which the associated luminous element 20, 22, 24, 26 emits, but to otherwise largely transmit. For this purpose, notch filters can be used in the medium wavelength range, which can have, for example, transmission spectra 100 and 102. At spectral edges, high-pass or low-pass filters can be used instead of notch filters (see transmission spectra 98 and 104).

Due to the specific transmission spectra 98, 100, 102, 104 of the crossed beam splitters 30, 32, light from the fifth luminous element 28 is spectrally clipped. It may therefore be expedient, in the manner already mentioned, to supplement the light blocked by the beam splitters 30, 32 in a targeted manner by means of the luminous elements 20 and 24, if necessary also 22 and/or 26. This allows for supplementing specifically in those spectral ranges in which the beam splitters 30, 32 absorb and/or reflect light from the fifth luminous element 28, but in any case do not transmit it to the optical interface 16. The additionally used luminous elements 20, 24 and, if applicable, 22 are preferably operated with reduced power or with adjusted power. The aim here may be to at least largely restore the original spectrum of the fifth luminous element 28.

In some embodiments, the fifth luminous element 28 may alternatively be a green luminous element or, more generally, a colored luminous element that emits primarily in the spectral range that the at least one beam splitter 30, 32 transmits. For example, in such embodiments, the fifth luminous element 26 may be an LED having an emission peak at about 530 nm. A green laser diode can also be used for this purpose. Here, it can be provided that color mixing takes place in white light mode and, in particular, that no individual white light source such as a white light LED is used, but that white light from separate luminous elements is specifically mixed.

It shall be understood that in case of suitable dyes, such a green luminous element can also be used in fluorescence mode. Alternatively or additionally, it could be used in multispectral mode.

The illumination unit 18 defines a common optical path 54 into which emitted light from the luminous elements 20, 22, 24, 26, 28 can be coupled. The common optical path 54 extends from the output side 44 of the second crossed beam splitter 32 to the optical interface. In the present case, the common optical path 54 is arranged coaxially with the fifth luminous element 26.

In the embodiment shown, the luminous elements 20, 26 of the first group are arranged such that light emitted by the luminous elements 20, 26 travels through a light path of at least substantially the same length from the relevant luminous element 20, 26 to the optical interface 16. The luminous elements 20, 26 of the first group each have a light-emitting surface 56, 58. The light-emitting surfaces 56, 62 are arranged equidistantly with respect to the common optical path 54. In the present case, this is achieved in that the two luminous elements 20, 26 are arranged at the same distance from the beam splitter 32 assigned to them (in the present case, by way of example, the second beam splitter 32), in particular from its opposite input sides 38, 40. The light is coupled by the crossed beam splitter 32 into the common optical path 54.

The beam splitters 30, 32 are in particular arranged such that light-emitting surfaces 56, 58, 60, 62, 64 of the luminous elements 20, 22, 24, 26, 28 are each arranged equidistantly with respect to their associated crossed beam splitter 30, 32.

By using crossed beam splitters 30, 32 and luminous elements 20, 22, 24, 26, 28 that can be used together for different modes, the illumination unit 18 or the illumination device 12 has a high degree of compactness. In addition, the equidistant arrangement ensures that no spectral shifts occur when the imaging unit 14 or its light guide is rotated relative to the optical interface 16.

It shall be understood that a different number of luminous elements 20, 22, 24, 26, 28 and/or a different number of crossed beam splitters 30, 32 can be used. The use of crossed beam splitters 30, 32 has proven to be particularly useful. In other embodiments, however, other types of beam splitters and/or other optical elements can be used to couple light from the luminous elements 20, 22, 24, 26, 28 into the optical interface 16.

FIG. 4 shows a schematic representation of the imaging device 10. The imaging unit 14 is optically coupled to the optical interface 16, for example via a light guide 106 such as at least one optical fiber.

The imaging device 10 has a controller 66 which is configured to automatically match an operating state of the imaging unit 14 and an illumination mode of the illumination unit 18 to one another. In the present case, a user can specify the operating mode of the imaging unit 14 by means of a user action. The controller 66 then adjusts the appropriate illumination mode of the illumination unit 18. Alternatively or additionally, the user can adjust a specific illumination mode of the illumination unit 18 by means of a user action. The controller 66 can then adjust an appropriate operating mode of the imaging unit 14. The illumination device 12 and/or the imaging device 10 has, for example, a user interface via which the user can enter corresponding commands.

The imaging unit 14 comprises a camera unit 68 and a distal shaft 76. The distal shaft 76 is optically coupled to the camera unit 68. The camera unit 68 can have a connection for the distal shaft 76, wherein the distal shaft 76 can be optionally decouplable and couplable. The distal shaft 76 can also be permanently optically and/or mechanically coupled to the camera unit 68. The camera unit 68 is arranged proximally with respect to the shaft 76. The camera unit 68 comprises an imaging sensor system 108, in the present case, for example, a white light sensor 110 and a near-IR sensor 112. Generally speaking, the imaging sensor system 108 can have one or more at least spatially resolving light sensors/image sensors, for example at least one CMOS sensor and/or at least one CCD sensor. The shaft 76 comprises optical elements (not shown) by means of which light can be guided to the camera unit 68 in order to be able to optically capture the object to be imaged. The shaft 76 further comprises at least one light path 114, for example defined by a light guide such as an optical fiber, which leads to a distal portion 116 of the shaft 76 and by means of which the illumination light originating from the optical interface 16 of the illumination device 12 can be coupled out to the object to be imaged.

The camera unit 68 has different operating states, specifically for example at least one multispectral operating state and one fluorescence operating state and, in the present embodiments, additionally a white light operating state and possibly a hyperspectral operating state. The controller 66 automatically adapts the illumination mode of the illumination unit 18 to the current operating state of the camera unit 68. The controller 66 can set the image recording behavior of the camera unit 68. For example, the controller 66 can adjust the exposure time, sensitivity/amplification/gain and/or other operating parameters of the camera unit 68 or, in particular, its image capturing sensor system 108 and, if applicable, its optics, thereby defining different operating states of the imaging unit 14. In the present case, the controller 66 triggers the illumination unit 18 synchronously with the camera.

The imaging unit 14 comprises a filter unit 46 with optical filters 48, 50 52. Three optical filters are shown as an example, but it shall be understood that a different number can be used. The filter unit 46 can be switched between a multispectral mode and a fluorescence mode. Moreover, the filter unit 46 can additionally be switchable to a white light mode and/or a hyperspectral mode. The optical filters 48, 50, 52 can optionally be introduced into an observation beam path 70 of the camera unit 68, thereby defining different observation modes. In the present case, they define the operating states of the camera unit 68.

A basic imaging mode can be associated with a plurality of optical filters 48, 50, 52. In particular for fluorescence imaging, a different suitable optical filter can be used depending on the luminous element 20, 22, 24, 26, 28 used for excitation. For example, in the present case the first luminous element 20 (red) is combined with an optical filter that transmits wavelengths greater than 730 nm but blocks shorter wavelengths. This makes it possible in particular to ensure that only fluorescent light and not the excitation light itself is detected. For example, said optical filter can absorb at least in the 600 nm to 730 nm range. Furthermore, in the present case, for example, the second luminous element 22 (dark red) is combined with a filter which absorbs in the 700 to 850 nm range or which only transmits significantly above 850 nm.

The user can select a specific filter 48, 50, 52 and thereby directly selects an associated observation mode or operating state of the camera unit 68. For this purpose, the camera unit 68 has a filter sensor 72 which can automatically detect an optical filter currently introduced into the observation beam path 70. The user can thus manually introduce a selected filter 48, 50, 52 into the observation beam path 70. In the example shown, the optical filters 48, 50, 52 are mounted on a filter carrier 118. Said filter carrier 118 can be moved into different positions, whereby one of the optical filters 48, 50, 52 can be selected in each case. The filter sensor 72 then detects the currently selected optical filter 48, 50, 52. The controller can then determine the current operating state of the camera unit 68 and thus of the imaging unit 14 based on a sensor signal from the filter sensor 72 and automatically adapt the illumination mode of the illumination unit 18 accordingly. The user thus puts the entire imaging device 10 into the desired mode by a simple user action such as a manual selection of an optical filter 48, 50, 52. A user can basically combine different filters with different illumination modes to create different types of contrast.

In the illustrated case, the imaging unit 14 and in particular the shaft 76 comprises a broadband transmitting optics 77, which can be used uniformly in the different illumination modes. In the present case, the broadband optics 77 is designed for a spectral range of at least 400 nm to 1000 nm. It can be used uniformly for different illumination and/or observation spectral ranges.

In some embodiments, the imaging unit 14 can be configured as a stereoendoscope that comprises a stereoscopic ocular having two sides. Different optical filters can be connected upstream of these sides independently of one another, allowing different contrast images to be superimposed on one another.

In the following, in the context of further embodiments and modifications, the same reference signs as above will be used for identical or similar components. As regards their description, reference is generally made to the above embodiments, whereas primarily the differences between the embodiments will be explained below. Also, reference signs have been partially omitted in the following figures for reasons of clarity.

FIG. 5 shows a schematic representation of another embodiment of the imaging device 10. The imaging device 10 comprises an illumination device 12 having an optical interface 16, and an illumination unit 18 as well as an imaging unit 14 which is connected to the optical interface 16. The imaging unit 14 comprises a camera unit 68 having an automated filter unit 210. The automated filter unit 210 comprises a plurality of optical filters 48, 50, 52, which can be automatically introduced into an observation beam path 70 of the camera unit 68 in accordance with an observation mode specified by a user.

The automated filter unit 210 comprises a filter drive 212 which is configured to automatically move the optical filters 48, 50, 52 into or out of the observation beam path 70. The optical filters 48, 50, 52 can be mounted on a filter carrier 118 which is connected to the filter drive 212. The filter drive 212 can be configured to move, for example to shift and/or rotate and/or pivot, the filter carrier 118.

The imaging unit 14 has a user interface 214 by means of which the user can adjust a desired observation mode. For example, a desired position of the filter carrier 118 can be specified by means of the user interface 214.

The imaging unit 14 further has a controller 66. The controller 66 is coupled to the filter drive 212 and the user interface 214. The controller 66 is configured, in particular, to process a user specification of an observation mode and to control both the filter unit 210 and the illumination unit 18 in accordance with said user specification. The controller 66 can thus adjust an operating state of the imaging unit 14 and an illumination mode of the illumination unit 18 matched thereto in accordance with an observation mode selected by the user.

FIG. 6 shows a schematic representation of yet another embodiment of the imaging device 10. The imaging device 10 comprises an illumination device 12 having an optical interface 16, and an illumination unit 18 as well as an imaging unit 14 which is connected to the optical interface 16. The imaging unit 14 comprises a proximal base unit 310. The proximal base unit 310 is connected to the optical interface 16 of the illumination device 12. Illumination light generated by the illumination device 12 can thus be supplied to the proximal base unit 310. The imaging unit 14 further comprises a controller 66, which in some embodiments may be integrated in the base unit 310.

Optionally, different exchangeable shafts 312, 314 can be optically and electronically coupled to the proximal base unit 310. The base unit 310 has an interface 316 for coupling different exchangeable shafts 312, 314. Said interface 316 supplies the illumination light coming from the illumination device 12 to a coupled exchangeable shaft 312, 314. The interface 316 is further configured to electrically supply a coupled exchangeable shaft 312, 314 and/or to electronically connect it to the controller 66 of the imaging unit 14.

The exchangeable shafts 312, 314 each have an integrated camera 318, 320 and integrated optical filters 322, 324. The integrated cameras 318, 320 are configured as Tipcams. In the present case, the integrated camera 318 of a first exchangeable shaft 312 is configured for multispectral imaging. Furthermore, the integrated camera 310 of a second exchangeable shaft 314 is configured for fluorescence imaging. The optional optical filters 322, 324 can be adapted thereto.

In other embodiments, it is also possible to use exchangeable shafts that only include optical filters but no integrated camera. In that case, they can be couplable to a proximal camera unit. In some cases, the proximal camera unit can then be designed without an additional filter unit. The selection of a specific optical filter or a specific observation mode can be made by choosing a suitably equipped exchangeable shaft.

The controller 66 is configured to detect a coupled exchangeable shaft 312, 314. This can be done software-based, mechanically and/or through sensor detection. Depending on the detected exchangeable shaft 312, 314, the controller 66 can then determine in which operating state or in which observation mode the imaging unit 14 should be operated. The control unit 66 is also configured to adjust an illumination mode of the illumination unit 18. The control unit 66 is thus configured to adjust an illumination mode of the illumination unit 18 on the basis of the observation mode defined by a currently coupled exchangeable shaft 312, 314.

In the present case, the exchangeable shafts 312, 314 and the imaging device 10 are part of a medical imaging system 316. The medical imaging system 316 allows a user to select a suitable exchangeable shaft 312, 314, couple it to the base unit 310, and thereby define a mode for the entire imaging device 10. By simply changing the exchangeable shaft 312, 314, it is thus achieved that the illumination device 18 is automatically adapted to the image capture mode to be used.

FIG. 7 shows a schematic perspective representation of another embodiment of an imaging device 10′. The reference signs of this embodiment are provided with inverted commas for differentiation purposes. In this embodiment, the imaging device 10′ is designed as an exoscopic imaging device. It comprises an illumination device 12′ and an imaging unit 14′. Its basic functionality corresponds to that described above, but in this embodiment the imaging unit 14′ is configured as an exoscope.

Aspects of the above description can also be summarized or described as follows. FIG. 8 shows a schematic flow chart of a method for generating illumination light for an imaging unit 14 by means of an illumination device 12. The sequence of the method is also clear from the above explanations. The illumination device 12 comprises an optical interface 16 for the optical connection of an imaging unit 14, and an illumination unit 18 that is designed to provide illumination light to the optical interface 16, wherein the illumination unit 18 comprises a plurality of luminous elements 20, 22, 24, 26, 28 which are optionally activatable independently of one another and which are configured to emit light according to different emission spectra in order to provide the illumination light.

The method comprises a step S11 of at least temporarily activating a first group of the luminous elements 20, 22, 24, 26, 28 in order to provide illumination light for multispectral imaging. The method further comprises a step S12 of at least temporarily activating a second group of the luminous elements 20, 22, 24, 26, 28 in order to provide illumination light for fluorescence imaging. One of the luminous elements 20, 22, 24, 26, 28 is activated at least temporarily both when the first group of luminous elements 20, 22, 24, 26, 28 is activated at least temporarily and when the second group of luminous elements 20, 22, 24, 26, 28 is activated at least temporarily.

FIG. 9 shows a schematic flow chart of a method for operating an imaging device 10. The sequence of the method is also clear from the above explanations. An imaging device 10 having an imaging unit 14 is provided in a step S21. Illumination light is provided to the imaging unit 14 in a step S22. The provision of illumination light to the imaging unit 14 is carried out according to a method as described with reference to FIG. 8.

FIG. 10 shows a schematic flow chart of a method for operating an imaging device 10. The sequence of the method is also clear from the above explanations. The method comprises a step S31 of providing an illumination device 12 for providing illumination light for an imaging unit 14. The imaging unit 14 comprises an optical interface 16 for the optical connection of an imaging unit 14, and an illumination unit 18 that is designed to provide illumination light to the optical interface 16. The illumination unit 18 is multimodal and can be operated in a plurality of different illumination modes. The method further comprises a step S32 of providing an imaging unit 14 that can be connected to the optical interface 16 of the illumination device 12. The method moreover comprises a step S33 of automatically matching an operating state of the imaging unit 14 and an illumination mode of the illumination unit 18.

The following describes an aspect concerning the correction of object images according to depth information that can be obtained from calibration images. FIG. 11 shows a schematic representation of a medical imaging device 410 according to this aspect. The medical imaging device 410 can basically be constructed and/or designed like the imaging device 10 described above or else like the above imaging device 10′. In particular, reference is made to the above description regarding the operation of the components and details of the design of the imaging device 410. To explain the present aspect, it is expedient to describe the technical facts with reference to FIG. 11, which is to be understood purely schematically, and the other figures.

The imaging device 410 is an endoscope device in the specific example, but can also be an exoscope device and/or a microscope device. The imaging device 410 comprises an illumination unit 412 with at least one light source 414. The illumination unit 412 can, for example, be designed as described above with reference to the illumination device 12. For the following description, it is assumed that the illumination unit 412 is designed in this way. However, this is to be understood purely as an example. Basically, the illumination unit 412 is configured to provide illumination light 416, by means of which an object 418 to be imaged can be illuminated.

The imaging device 410 further comprises an image capturing unit 420 with a lens 442, which is only shown schematically, and with suitable image capturing sensors 444. The image capturing unit 420 is configured to detect object light 428 originating from the object 418. This can be remitted illumination light 416 and/or light emitted by the object 418, for example fluorescent light.

The image capturing sensor 444 is configured to be able to capture images in both the visible range and the near-infrared range. For example, the image capturing sensor 444 is sensitive at least in a range between 450 nm and 950 nm, in some embodiments in a range between 400 nm and 1000 nm.

In the following, it is assumed that the image capturing unit 420 in combination with the illumination unit 412 can be operated at least in a white light mode and in a fluorescence mode. In white light mode, broadband illumination light 416 is irradiated, for example by means of a white light luminous element, approximately at least in the range from 480 nm to 750 nm. Illuminating light 416 remitted by the object 418 is then observed. In fluorescence mode, however, illumination light 416 is irradiated with a specific wavelength that is suitable for exciting a fluorescent dye used. Furthermore, light emitted by the fluorescent dye is detected, which is emitted by the object 418 and in particular by excited dye molecules.

In the present case, the image capturing unit 420 is configured to capture stereo images. For this purpose, it can comprise suitable stereo optics and/or suitable stereo image capturing sensors 444.

The object 418 to be imaged is, for example, an anatomical structure, such as in a patient's cavity. The object 418 comprises a region 448 stained with a fluorescent dye. For example, indocyanine green is used as a dye. Furthermore, the object 418 comprises tissue 450 that covers the stained region 448. For example, the stained region 448 is a vessel and the tissue 450 is fatty tissue covering the vessel, although this is to be understood purely as an example. FIG. 12 shows a schematic representation of the imaging situation. A surface of the tissue 450 is located at a distance do from the imaging device 410, in particular from the lens 442 of the image capturing unit 420. The stained region 448 is located within the tissue 450 and is at a distance d₁ from the surface. In the following, it is assumed that illumination light is coupled out by the lens 442.

This has multiple implications for imaging. If illumination light 416 is used which has a low penetration depth into the object 418, it is substantially reflected and/or scattered by the surface of the tissue 450. The intensity of remitted light then depends on the distance do according to a distance law, approximately according to the well-known inverse square law. It is assumed that air is present in the range of the distance do, and is present in the cavity within which imaging is performed.

If illumination light 416 that can penetrate into the tissue 450 is used, and is suitable, for example, to reach the stained region 448 and excite dye molecules there to fluorescence, two effects must be taken into account. First of all, the radiated intensity is also subject to a distance law. Furthermore, within the tissue 450, an attenuation of the illumination light 416 takes place through interaction with the tissue 450. The intensity actually available for fluorescence excitation is thus lower than the intensity emitted by the illumination unit 412.

Object light 428 emitted from the stained region 448 is also subject to a certain attenuation in the tissue 450. In addition, the intensity of the emitted object light 428 also follows a distance law, where the total distance d₀+d₁ must be taken into account. The fluorescence intensity detectable by the image capturing unit 420 is thus lower than the fluorescence intensity emitted by the stained region 448.

As mentioned above, this results in a detectable intensity I_{detected} as follows:

$${I}_{detected} = I ( {Λ}_{0} ) / {( {d}_{0} + {d}_{1} )}^{α} \cdot exp ( - μ ( {Λ}_{0} ) \cdot {d}_{1} ) \cdot exp ( - μ ( {Λ}_{1} ) \cdot {d}_{1} ) \cdot R$$

where I_{detected} denotes the detected light intensity, I(λ₀) denotes the intensity of the incident light with wavelength λ₀, d₀ is a distance between the lens and the surface of the object to be imaged, d₁ is a distance between the surface of the object to be imaged and the region in the object to be imaged that is stained with fluorescent dye, α is an exponent that defines the inverse law and can be chosen, for example, to be 2, so as to calculate according to the inverse square law, μ(λ₀) is an attenuation factor for the attenuation of light of the wavelength λ₀ when it passes through the object to be imaged, and μ(λ₁) is an attenuation factor for the attenuation of light of the wavelength λ₁ when it passes through the object to be imaged.

To account for these effects and correct images accordingly, the imaging device 410 comprises an image correction unit 426. The functionality thereof is described below with reference to FIGS. 13 to 18.

In this example, two calibration images 422, 423 are first taken. These can be stereo images. A first calibration image 422 is obtained, for example, by illuminating with white light and detecting remitted light. Since white light has a low penetration depth into the object 418, the first calibration image 422 substantially shows a surface of the object 418. Light penetrating the object 418 and remitted from deeper layers can be neglected because the remitted intensity is significantly lower than the intensity remitted from the surface of the object 418 due to both the attenuation of the incident light and the attenuation of the remitted light in the tissue.

Furthermore, a second calibration image 423 is captured, for which light is irradiated with a wavelength at which the dye used can be excited. The second calibration image 423 is taken through a suitable observation filter and/or in a suitable wavelength range to detect fluorescent light. This originates from the stained region 448.

In this example, the image correction unit 426 is configured to determine a depth map 432, 434 from each of the two calibration images 422, 423. A stereo reconstruction algorithm is used for this purpose. The depth maps 432, 434 thus comprise information regarding an observed surface of the respective objects, i.e., in the case of the depth map 432, which is determined from the first calibration image, a surface of the object 418 being observed, and in the case of the depth map 434, which is determined from the second calibration image, a surface of the stained region 448 lying in the tissue 450.

From this, the distances d₀ and d₁ shown schematically in FIG. 12 can be determined. It is understood that such distances can be determined point by point. The depth maps 432, 434 contain, in particular, point-by-point depth information, so that a correction can be carried out on a pixel-by-pixel basis.

It should be noted that, due to scattering effects, the distance d₁ can be inferred from the second calibration image 423 taking into account a scattering factor. Due to scattering effects, the stereo reconstruction can result in a depth for the stained region 448 of d₀+x·d₁, where x is an empirically determined factor between 0 and 1. The factor x can be determined empirically, for example, by suitable calibration and then taken into account by the image correction unit 426 in order to determine the actual distance value d₁.

In this case, the correction comprises taking into account the above equation, i.e., distances and also attenuations are taken into account. It is then possible to determine the position of the stained region 448 in the tissue 450 by using the two depth maps 432, 434.

An object image 424 of the object 418 can then be taken. This can be based on multiple individual images and can, for example, be an overlay display on which a white light image and a fluorescence image are superimposed. Due to the described distance and attenuation effects, the stained region 448 in the object image 424 may appear significantly paler than the actual fluorescence emission. The image correction unit 426 is therefore configured to generate a corrected object image 430 in accordance with the correction. In the corrected object image 430, for example, the intensity of the fluorescent light originating from the stained region 448 is shown increased in accordance with the correction. The corrected object image 430 thus comprises at least one image region 436 which, in accordance with the correction, is enhanced and/or attenuated relative to another image region 437 with respect to at least one parameter, such as a hue, a brightness and/or a color saturation. The stained region 448 is then clearly visible to a user despite its location inside the tissue 450.

The corrected object image 430 can be output to a user via a (schematically illustrated) display 446 of the imaging device 410.

FIG. 19 shows another example of a corrected object image 430. In order to additionally provide the user with information about how deep the stained region 448 is located in the tissue 450, in this case the stained region 448 is displayed with a brightness/intensity corrected as described above, but in false colors according to a color scale 452. The color scale 452 contains information concerning a depth of the stained region 448 in the object 418. The color scale 452 can be displayed to the user so that the user can directly determine a specific depth from the displayed coloring of the stained region 448.

With reference to the above explanations regarding a multimodal illumination device 12, it is generally understood that, depending on the dye used, different wavelengths or wavelength mixtures can be used as illumination light 416 to acquire calibration images. Furthermore, illumination can also be carried out at a wavelength at which dye emission is expected in order to analyze the absorption/attenuation properties of the tissue under consideration. For example, if indocyanine green is used as a dye, a calibration image can be captured by irradiating light with a wavelength of approximately 940 nm (cf. fourth luminous element 26). In this case, it is not fluorescent light that is detected, as described above, but remitted light. A depth map determined in this way then provides information about the penetration depth and the absorption behavior of the tissue under consideration in the spectral range in which the dye emits during subsequent object imaging.

If, for example, Cy 5.5 is used as the dye, a calibration image can be taken to determine the absorption in the tissue that is relevant for its fluorescence. This image is irradiated with dark red light, for example with a wavelength of 770 nm (cf. second luminous element 22).

Alternatively or additionally, a calibration image that allows conclusions to be drawn about a surface of the object 418 can also be obtained by means of monochromatic and/or narrowband illumination. Multiple calibration images can also be captured in different spectral ranges to create spectrally dependent depth maps.

FIG. 20 illustrates a further use case. Here, the imaging device 410 is configured for multispectral and/or hyperspectral imaging. Such imaging can be used, for example, to measure certain tissue parameters, such as perfusion. For this purpose, the intensity of certain pixels associated with certain tissue types, such as blood vessels, is observed at suitable wavelengths. For example, perfusion measurements can be performed by comparing intensities at 680 nm and 930 nm. However, if the above effects, which affect the detected intensity, are not taken into account, incorrect parameters may be determined.

The imaging device 410 may therefore be configured to acquire multiple calibration images 422-1, 422-2, 422-3, 422-4 for different spectral ranges. These can be obtained, for example, by using one of the above-described luminous elements 20, 22, 24, 26 as an illumination light source in order to capture a corresponding calibration image. Preferably, stereo images are captured.

For example, depth maps 432-1, 432-2, 432-3, 432-4 can be calculated from the calibration images 422-1, 422-2, 422-3, 422-4 using stereo reconstruction. These are in turn assigned to certain spectral ranges. The depth maps 432-1, 432-2, 432-3, 432-4 contain information regarding an average penetration depth of the corresponding light. In addition, a white light image or an image using short-wavelength illumination light, such as blue light, can be taken as a further calibration image. From the further calibration image, a further depth map can be determined in the manner described above, which, due to the low penetration depth of the light, corresponds at least substantially to a surface of the object to be imaged. If the depth maps 432-1, 432-2, 432-3, 432-4 are each subtracted from this further depth map, the average penetration depth in the respective spectral ranges can be estimated. Accordingly, absorption losses in the tissue under consideration can then be taken into account.

Additionally or alternatively, as described above, a distance law can be incorporated to account for intensity losses due to distance from the illumination unit 412.

FIG. 21 shows a schematic flow chart of a method for medical imaging. The sequence of the method is also clear from the above explanations. The method is carried out, for example, by means of the imaging device 410. A step S41 comprises providing illumination light 416 for illuminating an object 418 to be imaged. A step S42 comprises capturing at least one calibration image 422, 423 of the object 418 to be imaged. A step S43 comprises capturing at least one object image 424 of the object 418 to be imaged. A step S44 comprises determining depth information from the calibration image 422, 423. A step S45 comprises determining a correction for the object image 424, wherein the correction comprises taking into account a location dependency of a light intensity of illumination light 416 and/or a distance dependency of a light intensity of object light 428 in accordance with the depth information. A step S46 comprises generating a corrected object image 430 in accordance with the correction.

FIG. 22 is a schematic representation of a computer program product 438 with a computer readable medium 440. The computer-readable medium stores program code which, when executed in a processor, is designed to carry out one and/or all of the described methods.

### LIST OF REFERENCE SIGNS

10 Imaging device
12 Illumination device
14 Imaging unit
16 Optical interface
18 Illumination unit
20 Luminous element
22 Luminous element
24 Luminous element
26 Luminous element
28 Luminous element
30 Beam splitter
32 Beam splitter
34 Input side
36 Input side
37 Input side
38 Input side
40 Input side
41 Input side
42 Output side
44 Output side
46 Filter unit
48 Filter
50 Filter
52 Filter
54 Optical path
56 Light-emitting surface
58 Light-emitting surface
60 Light-emitting surface
62 Light-emitting surface
64 Light-emitting surface
66 Controller
68 Camera unit
70 Observation beam path
72 Filter sensor
74 Display unit
76 Shaft
77 Optics
78 Lens
80 Lens
82 Lens
84 Lens
86 Lens
88 Lens
90 Beam splitter element
92 Beam splitter element
94 Beam splitter element
96 Beam splitter element
98 Transmission spectrum
100 Transmission spectrum
102 Transmission spectrum
104 Transmission spectrum
106 Light guide
108 Imaging sensor system
110 White light sensor
112 Near-IR sensor
114 Light path
116 Distal portion
210 Filter unit
212 Filter drive
214 User interface
310 Base unit
312 Exchangeable shaft
314 Exchangeable shaft
316 Imaging system
318 Camera
320 Camera
322 Filter
324 Filter
410 Imaging device
412 Illumination unit
414 Light source
416 Illuminating light
418 Object
420 Image capturing unit
422 Calibration image
423 Calibration image
424 Object image
426 Image correction unit
428 Object light
430 Corrected object image
432 Depth map
434 Depth map
436 Image region
437 Image region
438 Computer program product
440 Computer-readable medium
442 Lens
444 Image capturing sensor
446 Display
448 Stained region
450 Tissue
452 Scale

1-16. (canceled)

17. A medical imaging device, comprising:
an illumination unit having at least one light source configured to provide illumination light for illuminating an object to be imaged;
an image capturing unit configured to acquire at least one calibration image of the object to be imaged and to acquire at least one object image of the object to be imaged; and
an image correction unit, configured to:
determine depth information from the calibration image;
determine a correction for the object image, wherein the correction comprises taking into account a location dependency, of a light intensity of illumination light or a distance dependency of a light intensity of object light in accordance with the depth information; and
generate a corrected object image in accordance with the correction.

18. The medical imaging device of claim 17, wherein the location dependency of the light intensity of the illumination light is a distance dependency.

19. The medical imaging device of claim 18, wherein the depth information comprises at least one depth map.

20. The medical imaging device of claim 18, wherein an image capture of the calibration image comprises a detection of remitted light.

21. The medical imaging device of claim 18, wherein an image capture of the calibration image comprises a detection of fluorescent light.

22. The medical imaging device of claim 18, wherein the illumination unit is configured to be operable in different illumination modes in which illumination light can be provided in different spectral ranges, and wherein the image capturing unit is configured to capture a plurality of calibration images whose image capture is based on different illumination modes of the illumination unit.

23. The medical imaging device of claim 18, wherein the image capturing unit is configured to capture a plurality of calibration images in different spectral ranges simultaneously or sequentially.

24. The medical imaging device of claim 23, further comprising a plurality of optical filters, and wherein the plurality of calibration images in different spectral ranges are captured by passing the illumination light through the different optical filters.

25. The medical imaging device of claim 18, wherein the correction comprises a distance correction based on an inverse of a power of a length of a light path.

26. The medica imaging device of claim 25, wherein the correction based on an inverse power of a length is a length of a light path between the image capturing unit and the object to be imaged or a light path within the object to be imaged.

27. The medical imaging device of claim 18, wherein the correction comprises an absorption correction based on an attenuation of light along a light path having a length within the object to be imaged.

28. The medical imaging device of claim 27, wherein the attenuation is an exponential attenuation.

29. The medical imaging device of claim 28, wherein the absorption correction takes into account an attenuation of illumination light or an attenuation of object light.

30. The medical imaging device of claim 18, wherein the image correction unit is configured to determine spatial or spectral properties of the illumination unit from the at least one calibration image and to take the determined spatial or spectral properties into account during the correction.

31. The medical imaging device of claim 30, wherein the spatial or spectral properties are inhomogeneities.

32. The medical imaging device of claim 18, wherein the image correction unit is configured to determine the depth information based on a stereo reconstruction.

33. The medical imaging device of claim 18, wherein, in the corrected object image, at least one image region is enhanced or attenuated in accordance with the correction relative to at least one other image region with respect to at least one parameter, such as a hue, a brightness or a color saturation.

34. The medical imaging device of claim 18, wherein the illumination unit or the image capturing unit is configured for multispectral imaging.

35. A method for medical imaging, comprising the steps of:
providing a medical imaging device, the medical imaging device comprising an illumination unit having at least one light source; an image capturing unit; and an image correction unit;
providing an illumination light for illuminating an object to be imaged;
capturing at least one calibration image of the object to be imaged;
capturing at least one object image of the object to be imaged;
determining depth information from the calibration image;
determining a correction for the object image, wherein the correction comprises taking into account a location dependency of a light intensity of illumination light or a distance
dependency of a light intensity of object light in accordance with the depth information; and
generating a corrected object image in accordance with the correction.

36. A program code which, when executed in a processor, is designed to perform the steps of
providing an illumination light for illuminating an object to be imaged;
capturing at least one calibration image of the object to be imaged;
capturing at least one object image of the object to be imaged;
determining depth information from the calibration image;
determining a correction for the object image, wherein the correction comprises taking into account a location dependency of a light intensity of illumination light or a distance dependency of a light intensity of object light in accordance with the depth information; and
generating a corrected object image in accordance with the correction.
